(19) United States
(12) Patent Application Publication
(10) Pub. No.: US 2014/0066481 A1
(43) Pub. Date: Mar. 6, 2014

(54) Water-Free Pharmaceutical Compositions Suitable for Local Anaesthetics

(71) Applicant: PHARMANEST AB, Solna (SE)

(72) Inventors: Mark Sundberg, Arsta (SE); Arne Brodin, Sodertalje (SE); Anders Carlsson, Stockholm (SE)

(73) Assignee: PHARMANEST AB, Solna (SE)

(21) Appl. No.: 13/638,533

(22) PCT Filed: Mar. 30, 2011
(86) PCT No.: PCT/EP2011/054928

§ 371 Date: Dec. 10, 2012

Related U.S. Application Data
(60) Provisional application No. 61/325,427, filed on Apr. 19, 2010.

(30) Foreign Application Priority Data
Apr. 1, 2010 (SE) 1050323-3

(51) Int. Cl. A61K 47/14; A61K 31/445; A61K 31/167

(52) U.S. Cl. CPC A61K 47/14; A61K 31/167; A61K 31/445
USPC 514/330; 514/626

(57) ABSTRACT

The present invention relates to a water-free pharmaceutical composition comprising one or more local anaesthetics in base form and which is suitable for topical administration. The composition further comprises a lipid vehicle comprising long-chain triglycerides (LCT) and at least 10% by weight of medium-chain monoglycerides (MCM) selected so the composition has an at least semi-solid appearance at the body temperature at the site of administration. The invention further relates to methods of producing and sterilizing the compositions.

### FIELD OF THE INVENTION

The present invention relates to water-free pharmaceutical compositions comprising local anaesthetics and lipid vehicle for topical administration. The pharmaceutical compositions can be used for reducing pain in connection with a large number of clinical conditions and clinical procedures.

### BACKGROUND TO THE INVENTION

Local anaesthetics are commonly used to inhibit nociceptive pain, and are usually administered by local injection. Pharmaceutical compositions for local injection normally contain local anaesthetics at a concentration of 1 to 2%.

In the preparation of pharmaceutical compositions for topical administration it is preferred to have the local anaesthetic present at a higher concentration, such as at a concentration of 5% or more.

Local anaesthetics of the amide type, ATC code N01 BB, are weak bases with a pKₐ of around 8. Consequently, in an aqueous solution at neutral pH these local anaesthetics are mostly present in their acid form. However, the acid form is charged and therefore less suitable to pass through biological membranes. In pharmaceutical compositions for topical administration it is therefore preferred to have the local anaesthetic present in its base form which can readily pass through biological membranes.

However, this leads to problems of the base form of the local anaesthetics relating to poor solubility and stability in aqueous solutions. This problem has been addressed for e.g. in EP 0833612 which discloses a pharmaceutical composition comprising an eutectic mixture of lidocaine base and prilocaine base. This mixture is in oil form at room temperature and can therefore be formulated as an emulsion. However, this eutectic mixture can only be obtained with few local anaesthetic agents with different melting point, exemplified by lidocaine base and prilocaine base.

EP 1629852 describes a system where the local anaesthetic is kept in a solution at acidic pH and only mixed with a buffering solution with high pH shortly before use, providing a solution of the local anaesthetic at a pH between 5.5 and 7. In this pH interval only a small portion of the local anaesthetic is present in the base form, the form that readily penetrates membranes. EP 0636020 describes a lipid carrier system comprising a mixture of an amphiphilic polar lipid such as a phospholipid or a galactolipid, and a non-polar lipid, such as a mono-, di-, or triglyceride, which spontaneously forms lipid particles, referred to as Biosomes. The system is used to formulate lidocaine. EP 0 889771 describes water-free compositions of lidocaine comprising a triglyceride oil and an alcohol solubilizer. In order to provide fluid oily composition for topical use on the skin, a carrier oil based on triglyceride of medium length fatty acids (Miglyol 812) is suggested. WO 03/07785 discloses compositions of base form local anaesthetics with suggested improved efficacy and reduced adverse effects. These compositions included an oleaginous base vehicle and may in addition to solubility enhancers also include gelling agents, penetration enhancers and further additives.

While the discussed compositions may be useful as delivery systems for base forms of local anaesthetics made effective topically at the surface of the body, they leave no provisions of how to obtain a formulation that is both suitably cohesive to safely exert the correct anaesthetic effect and yet be conveniently administrable by conventional invasive tools in order to be suitable for administration to an internal body site. The present invention aims at providing such suitable stable water-free pharmaceutical compositions comprising one or more local anaesthetics at a concentration sufficiently high to be able to provide pain relief following topical administration also at internal body sites, such as body cavities.

### SUMMARY OF THE INVENTION

The present invention relates to a water-free anaesthetic pharmaceutical composition comprising one or more local anaesthetics in an anaesthetically effective amount and a lipid vehicle comprising long-chain triglycerides (LCT) and monoglycerides. The monoglycerides preferably comprise fatty acids with medium length, preferable the monoglycerides comprise a mixture of fatty acids dominated by C8 and C10 fatty acids. The relative amount of monoglycerides and long-chain triglycerides adapted provides the composition with the combined properties of being ejectable with different applicators including standard cannulas at room temperature, being sufficiently cohesive at its administration site, typically at body temperature inside the body so it exerts its predetermined, correct anaesthetic effect in a controlled way; and being sterilizable with sterile filtration at a sufficiently low temperature of about 50 to 55° C. In other terms, the compositions have a semi-solid appearance at body temperature and the appearance of a liquid at 50-55° C. The compositions can further comprise solubilizers, stabilizers, preservatives and other conventional additives. The inventive compositions and their advantages will be further described and exemplified in the following sections.

### DESCRIPTION OF THE INVENTION

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of ±2% of the given value, preferably ±5%, and most preferably ±10% of the numeric values, where applicable.

The present invention relates in a first general aspect to a water-free anaesthetic pharmaceutical composition comprising one or more local anaesthetics in an anaesthetically effective amount and a lipid vehicle comprising long-chain triglycerides (LCT) and at least 10% by weight of medium-chain monoglycerides (MCM). The lipid vehicle is purposefully selected so that the composition obtains a solid fat content (SFC) that is 40 to 60% at room temperature, 10 to 40% at body temperature, and essentially 0% at a temperature exceeding 50° C.

By a water-free composition is meant a composition which is essentially free of water. The composition can comprise small amounts of water emanating from the lipid vehicle, the solubilizers or any anaesthetics added as a hydrate, or other components added to the composition. No additional water is added during preparation of the composition. Accordingly, the water-free composition according to the invention comprises typically less than 1% by weight of water, and more typically less than 0.1% by weight of water. The lipid vehicle can further comprise residual organic solvents, such as ethanol, from the preparation procedure.

In the context of the present “long-chain triglycerides” or “LCT” means a triglyceride fat of natural, semi-synthetic or synthetic origin. The natural fat, for example originating from a variety of sources like palm oil, soybean oil, olive oil, coconut oil, etc., which can be processed with conventional methods. Typically an LCT used with the present invention retains the fatty acid pattern of the natural oil and it may include both long and medium-chain fatty acids and amounts of diglycerides and monoglycerides. Typical brands used or useful with the inventive compositions, also used in cosmetic preparations, are for example Lipex Shea, Lipex Sheasoft, Lipex Cocoasoft and Akosoft 36. There are also long-chain triglycerides which are unsuitable to use, such as Adeps Solidus, due to a too low SFC at 37° C. The LCT content of the lipid vehicle provides suitable solid fat content levels and thereby sufficient cohesiveness at the body temperature of the administration site. It is generally contemplated that such characteristics depend on such parameters as chain length and saturation degree of the present fatty acids. The skilled person given the successful examples with the present invention would be able to produce a number of alternative LCTs of a pharmaceutical grade that will be suitable to enable the invention in its broad context.

Also in the context of the present invention, “medium-chain monoglycerides” or “MCM” are used as softness modifier of the compositions, for example to adapt the ejectability of the compositions to suitable cannulas and similar devices for application to site inside the body. MCM can comprise certain amounts of di- and triglycerides, but has a content of monoglycerides exceeding 40-50%. MCM useful with the present invention has dominating content of, preferably comprising, essentially straight, saturated fatty acids with a chain length of 8 to 10 carbon atoms. Suitable MCM has about 50 to about 90% 08 and about 10 to about 50% 010 fatty acids. In an example MCM has about 80% 08 and 20% 010 fatty acids. Suitable brands of MCM are Akoline MCM and Capmul MCM.

Solid fat content (SFC), the percentage of solid at a certain temperature determined by pulse NMR, is the ratio of the response from the hydrogen nuclei in the solid phase and that from all the hydrogen nuclei in the sample. Standard methods are described by American Oil Chemists' Society (AOCS).

It is preferable that the lipid vehicle admits that the pharmaceutical composition is ejectable with a cannula with an inner diameter as fine as 15 Gauge, or ejectable with other applicators having a similarly fine tip/needle at room temperature such as an inner diameter of a few millimetres.

In a preferred embodiment the lipid vehicle comprises 10 to 50% by weight of MCM. In a certain embodiment the lipid vehicle comprises about 50% by weight LCT and about 50% by weight MCM. Examples of such embodiments are described in the following detailed description. In the mentioned embodiments, it is preferred that MCM essentially comprises 08 and 010 fatty acids, preferably about 80% 08 fatty acids and about 20% 010 fatty acids.

The pharmaceutical compositions according to invention can further comprise one or more solubilizers in an amount of between about 0 and about 30% by weight, such as in amount of between about 0 and about 25%, most preferably in an amount of between about 0 and about 10%.

The solubilizer can be select from suitable lower alcohols such as ethanol, propanol, isopropanol, propylene glycol and benzyl alcohol; glycerol formal, glycofural, polysorbate 80, decanol, 2-ethyl hexanol, ethyl acetate, butyl acetate, ethyl hexanoic acid, lactic acid, caproic acid, peppermint oil and dimethyl sulphoxide. Most preferably the solubilizer is benzyl alcohol.

The one or more local anaesthetics are preferably present in an amount of between about 0.1 and about 20% by weight, more preferably in an amount of between about 0.5 and about 10% by weight and most preferably in an amount of between about 2 and about 10% by weight.

The local anaesthetic to be used in the pharmaceutical compositions according to the invention can be any local anaesthetic. Preferably the local anaesthetic is a local anaesthetic of the amide type, ATC code N01 BB or a local anaesthetic of the ester type, ATC code N01 BA. Most preferably the local anaesthetic of the amide type is selected from the group consisting of lidocaine, prilocaine, mepivacaine, ropivacaine, bupivacaine, and levobupivacaine. Most preferably the local anaesthetic of the ester type is selected from benzocaine, tetracaine, and chloroprocaine. According to one embodiment the pharmaceutical composition according to the invention comprises one or more long acting local anaesthetics such as ropivacaine, bupivacaine, and levobupivacaine. According to another embodiment the pharmaceutical composition according to the invention comprises one or more short acting local anaesthetics such as lidocaine, prilocaine, and mepivacaine. Preferred water-free compositions according to the invention include lidocaine or ropivacain in 2 to 10% by weight in a lipid vehicle comprising essentially equal amounts (% by weight) of LCT and MCM with the above mentioned requirements of SFC.

In one special embodiment, the compositions comprise about 1-15% by weight, preferably about 5% by weight of ropivacaine and the lipid vehicle comprises about 50% weight of LCT (such as Lipex Cocoasoft) and about 50% MCM (such as Akoline MCM) and are semi-solid at body temperature, wherein the LCT has a SFC of about 25-35% SFC at body temperature, while the composition is ejectable at ambient temperature.

In another special embodiment, the compositions comprise about 1-15% by weight, preferably about 5% by weight of lidocaine and the lipid vehicle comprises form about 50 to about 75% by weight of LCT (such as Lipex Shea) and from about 20 to about 50% by weight of MCM (such as Akoline MCM) wherein the LCT has a SFC of about 5-15% SFC at body temperature, while the composition is ejectable at ambient temperature.

In still another embodiment, the compositions comprise about 1-15% by weight, preferably about 5% by weight of lidocaine and the lipid vehicle comprises form about 75 to about 95% by weight of LCT (such as Akosoft 36 or Lipex Cocoasoft or mixtures thereof) and from about 5 to about 25% by weight of MCM (such as Akoline MCM) wherein the LCT has a SFC of about 5 to 15% SFC at body temperature, while the composition is ejectable at ambient temperature.

In another aspect, the present invention relates a method of preparing a water-free pharmaceutical composition of one or more local anaesthetics of a form defined above, such as especially lidocaine or ropivacine without using any solubilizers. This method generally comprises the provision of a lipid vehicle comprising long-chain triglycerides (LCT) and at least 10% by weight of medium-chain monoglycerides (MCM) as defined in foregoing sections, wherein the composition has a solid fat content (SFC) that is 40 to 60% by weight at room temperature, 10 to 40% at body temperature, and essentially 0% at a temperature exceeding 50° C.; heating the so provided lipid vehicle to a temperature where it has essentially 0% SFC and a liquid appearance; and admixing the so heated lipid vehicle with a preparation, free from any solubilizer, of one or local anaesthetics and thereby obtaining an anaesthetically effective composition. The method can further comprise the step of subjecting the resulting mixture, under conditions where it has essentially 0% SFC, to pass through a filter having a pore size sufficient to sterilize the mixture. Advantageously, the carefully selected lipid vehicle having the specified SFC at critical temperatures admits a simple production process free from solubilising agents that depends on simply melting solid local anaesthetic agent in the lipid vehicle. The production method further allows for an advantageous sterilization process that is performed at so low temperatures as below 60° C., preferably 50 to 55° C. The sterilization step thereby can rely on conventional sterile filtering techniques with hydrophobic filters of pore sizes between 0.1 and 0.5 μm. In order to support the admixing step of lipid vehicle and local anaesthetic a mixing aid, as described in the foregoing can be added to the composition. The mixing aid is preferably ethanol or another biocompatible agent that can be evaporated at low temperature such as isopropanol, hexane or similar solvents.

In still another aspect the present invention extends to a method of sterilizing a water-free pharmaceutical composition comprising an at least partially lipid soluble pharmaceutical agent. This method aspect relies on that the lipid vehicle has the earlier described features and advantages; and providing a composition comprising the pharmaceutical agent and a lipid vehicle comprising long-chain triglycerides (LCT) and at least 10% by weight of medium-chain monoglycerides (MCM), wherein the composition has a solid fat content (SFC) that is 40 to 60% at room temperature, 10 to 40% at body temperature, and essentially 0% at a temperature of below 60° C., preferably at a temperature of about 50 to 55° C.; heating the composition the composition to a temperature where it has essentially 0% SFC; and subjecting so heated composition to pass through a hydrophobic filter having a pore size sufficient to sterilize the mixture, such as filters of pore sizes between 0.1 and 0.5 μm. The lipid vehicle suitably can be anyone exemplified and regarded suitable for administration for the typical uses of the invention in the foregoing sections.

It is evident that the invented compositions allow for a desirably simplified sterilization process that effectively enables their use as products approved beyond conventional topical use on the body surface. For this reason, the inventive compositions enable also such heat degradation liable systems to become more widely useful, which otherwise would decompose with unpredictable and potentially hazardous consequences when steam sterilized (autoclaved, typically at 121° C. for up to 10-15 minutes for aqueous systems and at considerably harsher conditions for non-aqueous systems, such as oil-based systems).

Accordingly, the pharmaceutical composition according to the invention can be formulated for topical administration on any mucosal tissue or any internal body sites, such as but not limited to, oral, nasal, intravaginal, intracervical, pericervical, intrauteral, and intrarectal administration. These compositions can be formulated for dermal administration on healthy, diseased and/or injured skin. Dermal administration can be made directly from the container, by hand, or by means of or together with patches, bandages and wound dressings.

The compositions according to the invention can be administrated by means of a syringe, preferably with needle/tip as fine as 18 Gauge. The syringe can be further provided with an applicator. The applicator can be in the form of a tube.

The pharmaceutical compositions according to the present invention can be used for reducing pain in connection with various clinical conditions and clinical procedures. Accordingly, in one aspect the present invention provides methods for reducing pain in connection with clinical conditions and clinical procedures comprising the administration of a pharmaceutical composition according to the invention. Clinical conditions are exemplified by, but not limited to, wound healing, especially burn wounds, skin ulcers, haemorrhoids, anal fissures, herpes zoster, herpes simplex infections, especially herpes labialis and herpes genitalis. Clinical procedures are exemplified by, but not limited to, obstetric procedures, such as during labour, gynaecological procedures, such as, abortions and application of intra uterine devices, hysteroscopy, in vitro fertilization, spontaneous and legal abortions, and general vaginal examination, dental procedures, surgical procedures, such as skin grafting.

The compositions according to the present invention surprisingly well meet a number of requirements difficult to reach with conventional vehicles for local administration. More specifically, the compositions are surprisingly well adapted to be administrable and be effective at internal body cites such as the cervix and the uterus for which there are no commercially available satisfying long term acting anaesthetic compositions based on controlled slow release of the anaesthetically active agent. The inventive compositions exhibit an excellent stability during manufacturing and storage. They can be readily sterilized at low temperatures so to maintain the integrity of its components. Further, the compositions exhibit excellent compliance as they can be administered with standard cannulas. It is of further importance that the inventive compositions exhibit a suitable cohesiveness at body temperature in order to establish that the anaesthetic effect is correctly exerted at the desired body site.

### DETAILED AND EXEMPLIFYING DESCRIPTION OF THE INVENTION

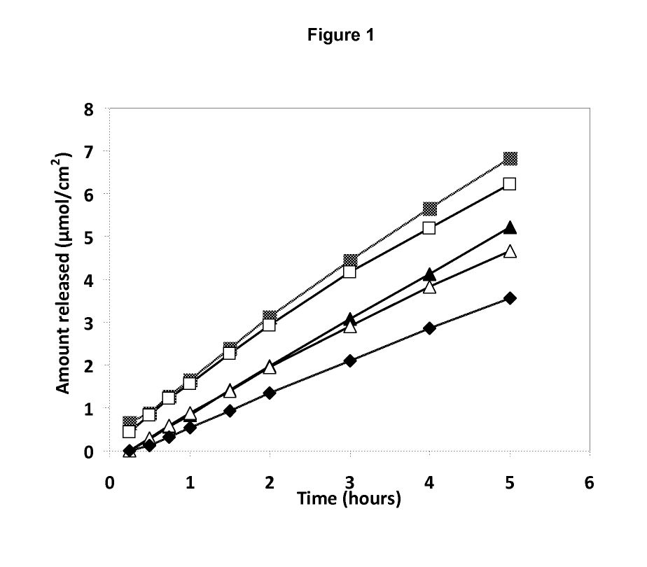

FIG. 1 is a graph illustrating in vitro release of lidocaine from pharmaceutical compositions. All compositions comprised 5% lidocaine. The water-free lipid vehicle comprised -▪- 25% MCM, 75% Lipex Shea; -□- 26% MCM, 74% Lipex Shea; -▴- 50% MCM, 50% MCT; -Δ- 49% MCM, 51% Lipex Shea; -♦- 100% MCM.

### EXAMPLES

### Example 1

### Procedure for Making and Evaluating Lipid-Based Formulations Containing Local Anaesthetics

The following procedure was adopted for the preparation of ropivacaine and lidocaine-containing lipid formulations, batch size 20-200 g, to be used for testing in vitro.

The desired amount of local anaesthetic was weighed in a 250 ml (1000 ml) round-bottomed flask. To every gram of local anaesthetic a volume of about 20-25 ml of absolute ethanol was added. The mixture was treated in an ultrasonication bath, set at 50 to 65° C. depending on the solid fat content of the lipids used, until a clear liquid was obtained. This was normally accomplished within a few minutes. The lipid ingredients (see below) were then added and the resulting mixture was treated in the ultrasonication bath until a clear, homogenous liquid was obtained.

The alcohol was evaporated from the liquid on a rotary evaporator at a pressure of about 25 mbar and a temperature of about 40 to 60° C. until the weight of the flask was more or less constant. In practice, the remaining ethanol content was always less than 1%. The evaporation time was normally 30 min. If necessary, the flask was additionally heated using a hot-air gun in order to prevent solidification during the evaporation. The oily liquid formulation was then transferred to clear glass vials and stored at room temperature unprotected from light until evaluation.

The evaluation comprised observation of the physical stability at room temperature over time. A physically stable formulation does not undergo any physical changes in appearance when stored and thus remains clear on standing (provided that the lipids used are in a liquid state at room temperature). Possible physical instability is haziness, precipitation of aggregates, and subsequent sedimentation and/or phase separation of two or more liquid phases, and/or change of colour.

The following lipids were used:

Akoline medium-chain monoglyceride (MCM)
Akosoft 36 long-chain triglyceride (LCT)
Lipex Shea long-chain triglyceride (LCT)
Lipex Shea soft long-chain triglyceride (LCT)
Lipex Cocoasoft long-chain triglyceride (LCT)
Akomed R medium-chain triglyceride (MCT)
(all from AarhusKarlshamns Sweden AB, Karlshamn, Sweden)
Capmul medium-chain monoglyceride (MCM)
(from Abitec Corp.)
Adeps Solidus hard (hydrogenated) long-chain triglyceride (LCT)
(from Apoteket AB, Sweden).

Akoline MCM has a melting point of about 25° C.

Akosoft 36 has a typical solid fat content of 40% at 20° C., 7% at 30° C. and 1% at 40° C.

Lipex Shea has a typical solid fat content of 33-37% at 20° C.

Lipex Sheasoft has a typical solid fat content of 30-35% at 20° C.

Lipex Cocoasoft has a typical solid fat content of 62-66% at 20° C.

Akomed R has a solid fat content of 0% at 20° C.

Adeps Solidus has a solid fat content of 100% at 20° C.

See also Table 1.

TABLE 1
Typical SFC content in percent for different lipids

| | SFC at | SFC at | SFC at | SFC at | SFC at | SFC at | |
|---|---|---|---|---|---|---|---|
| | 10° C. | 20° C. | 25° C. | 30° C. | 35° C. | 40° C. | Method* |
| Lipex Shea | 48-51 | 33-37 | | 7-10 | 0-2 | 0-1 | P- |
| | | | | | | | IUPAC |
| Lipex Sheasoft | 41-48 | 30-35 | 27-31 | 22-27 | 15-19 | 10-13 | NMR |
| | | | | | | | 26-40 |
| Lipex Cocoasoft | 70-74 | 62-66 | 56-60 | 44-48 | 33-37 | 23-27 | NMR |
| | | | | | | | 20-40 |
| Akosoft 36 | 73-74 | 40-42 | | 7-9 | 3-4 | 0-2 | P- |
| | | | | | | | IUPAC |

*P-IUPAC = IUPAC 2.150(a), NMR 26-40 & NMR 20-40 = IUPAC 2.150(b); NMR 26-40: 26° C. for 40 h, NMR 20-40: 20° C. for 40 h.

Examples of stable formulations of ropivacaine are presented in Table 2.

TABLE 2
Compilation of stable lipid-based ropivacaine formulations

| | | | Appearance | Appearance |
|---|---|---|---|---|
| | | | after 1 | after 15 |
| | | Observation | month | months |
| Ropivacaine | Lipid vehicle | at | at room | at room |
| (mg/g) | (% by weight) | preparation | temperature | temperature |
| 15 | 100% Akomed | Solution → | Unchanged | Unchanged |
| | R MCT | oil | | |
| 16 | 95% Akomed | Solution → | Unchanged | Unchanged |
| | R MCT, | oil | | |
| | 5% benzyl | | | |
| | alcohol | | | |
| 29.5 | 100% Akoline | Solution → | Unchanged | Unchanged |
| | MCM | oil | | (except for a |
| | | | | change in |
| | | | | colour) |
| 31 | 51% Akoline | Solution → | Unchanged | Unchanged |
| | MCM, | oil | | (except for a |
| | 49% Akomed | | | change in |
| | R MCT | | | colour) |
| 30 | 90% Akomed | Solution → | Unchanged | Unchanged |
| | R MCT, | oil | | (except for a |
| | 10% benzyl | | | change in |
| | alcohol | | | colour) |
| 40 | 100% Akoline | Solution → | Unchanged | Unchanged |
| | MCM | oil | | (except for a |
| | | | | change in |
| | | | | colour) |
| 40 | 50% Akoline | Solution → | Unchanged | Unchanged |
| | MCM, | oil | | (except for a |
| | 50% Akomed | | | change in |
| | R MCT | | | colour) |
| 51 | 100% Akoline | Solution → | Unchanged | Unchanged |
| | MCM | oil | | (except for a |
| | | | | change in |
| | | | | colour) |
| 60 | 100% Akoline | Solution → | Unchanged | Unchanged |
| | MCM | oil | | (except for a |
| | | | | change in |
| | | | | colour) |
| 75 | 89% Akoline | Solution → | Unchanged | Unchanged |
| | MCM, | oil | | (except for a |
| | 10% benzyl | | | change in |
| | alcohol | | | colour) |
| 74 | 51% Akoline | Solution → | Unchanged | Unchanged |
| | MCM, | ointment | | (except for a |
| | 49% Lipex | | | change in |
| | Shea LCT | | | colour) |
| 75 | 52.5% Akoline | Solution → | Unchanged | Unchanged |
| | MCM, | ointment | | (except for a |
| | 47.5% Lipex | | | change in |
| | Shea soft LCT | | | colour) |
| 50 | 90% Lipex | Solution → | Unchanged | NA* |
| | Cocoasoft | ointment | | |
| | LCT, | | | |
| | 10% Akoline | | | |
| | MCM | | | |
| 50 | 50% Lipex | Solution → | Unchanged | NA* |
| | Cocoasoft | ointment | | |
| | LCT, | | | |
| | 50% Akoline | | | |
| | MCM | | | |

*No observation made

Examples of stable formulations of lidocaine are presented in Table 3.

TABLE 3
Compilation of stable lipid-based lidocaine formulations

| | | | Appearance | Appearance |
|---|---|---|---|---|
| | | | after 2 | after 12 |
| | | Observation | weeks | months |
| Lidocaine | Lipid vehicle | at | at room | at room |
| (mg/g) | (% by weight) | preparation | temperature | temperature |
| 51 | 100% Akoline | Solution → | Unchanged | Unchanged |
| | MCM | oil | | |
| 51 | 50% Akoline | Solution → | Unchanged | Unchanged |
| | MCM, | oil | | |
| | 50% Akomed | | | |
| | R MCT | | | |
| 100 | 50% Akoline | Solution → | Unchanged | Unchanged |
| | MCM, | oil | | |
| | 50% Akomed | | | |
| | R MCT | | | |
| 50 | 51% Lipex | Solution → | Unchanged | Unchanged |
| | Shea LCT, | ointment | | |
| | 49% Akoline | | | |
| | MCM | | | |
| 50 | 75% Lipex | Solution → | Unchanged | Unchanged |
| | Shea LCT, | ointment | | |
| | 25% Akoline | | | |
| | MCM | | | |

### Example 2

### In Vitro Release of Local Anaesthetics from Pharmaceutical Compositions

Release of lidocaine from pharmaceutical compositions comprising 5% lidocaine prepared according to Example 1 was measured overtime.

Results are presented in FIG. 1. A steady release of lidocaine could be observed from the different pharmaceutical preparations.

### Example 3

### Evaluation of Appearance at 37° C.

Pharmaceutical compositions comprising 5% lidocaine or 5% ropivacaine were prepared according to Example 1. Examples of stable compositions are presented in Table 4. After preparation the formulations were equilibrated at 37° C. using thermostated water bath.

TABLE 4
Stable lipid-based lidocaine and ropivacaine formulations -
appearance at 37° C. and ejectability at room temperature (about 25° C.)

| | | Observation | | Ejectability |
|---|---|---|---|---|
| | Lipid vehicle | at | Appearance | at room |
| | (% by weight) | preparation | at 37° C. | temperature |
| Lidocaine | | | | |
| (mg/g) | | | | |
| 50 | 51% Lipex | Solution → | Semi-solid | Possible to |
| | Shea LCT, | ointment | | eject through |
| | 49% Akoline | | | the tip of a |
| | MCM | | | syringe |
| 50 | 50% Adeps | Solution → | Clear liquid | Too hard to |
| (compar- | Solidus LCT, | ointment | | eject through |
| ative) | 25% Akomed | | | the tip of a |
| | R MCT, | | | syringe |
| | 25% Akoline | | | |
| | MCM | | | |
| 50 | 50% Adeps | Solution → | Clear liquid | Too hard to |
| (compar- | Solidus LCT, | ointment | | eject through |
| ative) | 50% Akoline | | | the tip of a |
| | MCM | | | syringe |
| 50 | 75% Lipex | Solution → | Semi-solid | Possible to |
| | Shea LCT, | ointment | | eject through |
| | 25% Akoline | | | the tip of a |
| | MCM | | | syringe |
| 50 | 50% Akoline | Solution → | Clear liquid | ND |
| (compar- | MCM, | ointment | | |
| ative) | 25% Lipex | | | |
| | Shea LCT, | | | |
| | 25% Adeps | | | |
| | Solidus LCT | | | |
| 50 | 74% Lipex | Solution → | Semi-solid | Possible to |
| | Shea LCT, | ointment | | eject through |
| | 26% Akoline | | | the tip of a |
| | MCM | | | syringe |
| 50 | 75% Akosoft | Solution → | Semi-solid | Possible to |
| | 36 LCT, | ointment | | eject through |
| | 25% Akoline | | | the tip of a |
| | MCM | | | syringe |
| 50 | 80% Akosoft | Solution → | Semi-solid | Possible to |
| | 36 LCT, | ointment | | eject through |
| | 20% Akoline | | | the tip of a |
| | MCM | | | syringe |
| 50 | 90% Akosoft | Solution → | Semi-solid | Possible to |
| | 36 LCT, | ointment | | eject through |
| | 10% Akoline | | | the tip of a |
| | MCM | | | syringe/18 G |
| | | | | cannula |
| 51 | 95% Akosoft | Solution → | Semi-solid | Possible to |
| | 36 LCT, | ointment | | eject through |
| | 5% Akoline | | | the tip of a |
| | MCM | | | syringe/18 G |
| | | | | cannula |
| 50 | 90% Lipex | Solution → | Semi-solid | Possible to |
| | Cocoasoft | ointment | | eject through |
| | LCT, | | | the tip of a |
| | 10% Akoline | | | syringe |
| | MCM | | | |
| 50 | 45% Lipex | Solution → | Clear liquid | Difficult to |
| (compar- | Cocoasoft | ointment | | eject through |
| ative) | LCT, | | | the tip of a |
| | 45% Adeps | | | syringe |
| | Solidus LCT, | | | |
| | 10% Akoline | | | |
| | MCM | | | |
| 50 | 51% Lipex | Solution → | Semi-solid | Possible to |
| | Cocoasoft | ointment | | eject through |
| | LCT, | | | the tip of a |
| | 49% Akoline | | | syringe |
| | MCM | | | |
| 51 | 46% Lipex | Solution → | Semi-solid | Possible to |
| | Cocoasoft | ointment | | eject through |
| | LCT, | | | the tip of a |
| | 44% Akosoft | | | syringe |
| | 36 LCT, | | | |
| | 10% Akoline | | | |
| | MCM | | | |
| 50 | 46% Lipex | Solution → | Semi-solid | Possible to |
| | Shea LCT, | ointment | | eject through |
| | 44% Lipex | | | the tip of a |
| | Cocoasoft | | | syringe |
| | LCT, | | | |
| | 10% Akoline | | | |
| | MCM | | | |
| Ropivacaine | | | | |
| (mg/g) | | | | |
| 50 | 90% Lipex | Solution → | Semi-solid | Too hard to |
| | Cocoasoft | ointment | | eject through |
| | LCT, | | | the tip of a |
| | 10% Akoline | | | syringe |
| | MCM | | | |
| 50 | 50% Lipex | Solution → | Semi-solid | Possible to |
| | Cocoasoft | ointment | | eject through |
| | LCT, | | | the tip of a |
| | 50% Akoline | | | syringe |
| | MCM | | | |

### Example 4

### Preparation of Two Compositions without the Use of Ethanol

The lipid ingredients MCM and Lipex Cocoasoft (for compositions, see Table 5) were weighed in a beaker and melted at 60° C. Lidocaine was weighed and dissolved in the melted lipid mixture (50-60° C.).

TABLE 5

| Lidocaine | Lipid vehicle |
|---|---|
| 1.0 g | 9.5 g Akoline MCM |
| | 9.5 g Lipex Cocoasoft LCT |
| 1.5 g | 14.25 g Capmul MCM |
| | 14.25 g Lipex Cocoasoft LCT |

### Example 5

### Evaluation of the Possibility to Sterilize by Filtration at Elevated Temperature

Any of the lipid-based formulation disclosed in Table 2-5, particularly those that are still in a semi-solid state at room temperature as well as at body temperature, are completely melted at about 50 to 55° C. (or any elevated temperature at which SFC is zero), maintained at this temperature and then filtered through a filter with 0.22 or 0.45 μm pore size in order to sterilize the composition. An example of a suitable filter is a hydrophobic Millex FG filter from Millipore Corp.

Although particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made to the invention without departing from the spirit and scope of the invention as defined by the claims.

1. A water-free anaesthetic pharmaceutical composition comprising
one or more local anaesthetics in an anaesthetically effective amount; and
a lipid vehicle comprising long-chain triglycerides (LCT) and at least 10% by weight of medium-chain monoglycerides (MCM), wherein the composition has a solid fat content (SFC) that is 40 to 60% at room temperature, 10 to 40% at body temperature, and essentially 0% at a temperature exceeding 50° C.

2. The pharmaceutical composition according to claim 1 ejectable with 15 Gauge cannula at room temperature.

3. The pharmaceutical composition according to claim 1, wherein the lipid vehicle comprises 10 to 50% by weight of MCM.

4. The pharmaceutical composition according to claim 1, wherein the MCM essentially comprises C8 and C10 fatty acids, preferably about 80% C8 fatty acids and about 20% C10 fatty acids.

5. The pharmaceutical composition according to claim 1, wherein the lipid vehicle comprises about 50% by weight LCT and about 50% by weight MCM.

6. The pharmaceutical composition according to claim 1, wherein the composition further comprises one or more solubilizers.

7. The pharmaceutical composition according to claim 1, wherein the one or more local anaesthetics are present in an amount of between 0.1 and 20% by weight, most preferably in an amount of between 2 and 10% by weight.

8. The pharmaceutical composition according to claim 1, wherein the one or more local anaesthetic is a local anaesthetic of the amide type, ATC code N01BB.

9. The pharmaceutical composition according to claim 8 wherein the local anaesthetic of the amide type is selected from the group consisting of lidocaine, prilocaine, mepivacaine, ropivacaine, bupivacaine, and levobupivacaine.

10. The pharmaceutical composition according to claim 1 wherein the one or more local anaesthetic is a local anaesthetic of the ester type, ATC code N01BA.

11. The pharmaceutical composition according to claim 10, wherein the local anaesthetic of the ester type is selected from the group consisting of benzocaine, tetracaine, and chloroprocaine.

12. The pharmaceutical composition according to claim 1 wherein the one or more local anaesthetic is a long acting local anaesthetic.

13. The pharmaceutical composition according to claim 12, wherein the long acting local anaesthetic is selected from the group consisting of ropivacaine, bupivacaine, and levobupivacaine.

14. The pharmaceutical composition according to claim 1, wherein the one or more local anaesthetic is a short acting local anaesthetic.

15. The pharmaceutical composition according to claim 14, wherein the short acting local anaesthetic is selected from the group consisting of lidocaine, prilocaine, and mepivacaine.

16. The pharmaceutical composition according to claim 6, wherein the water-free lipid vehicle comprises one or more solubilizers in an amount of between 0 and 30% by weight, preferably in amount of between 0 and 25% by weight, most preferably in an amount of between 0 and 10%.

17. The pharmaceutical composition according to claim 6, wherein the solubilizer is selected from the group consisting of suitable lower alcohols such as ethanol, propanol, isopropanol, propylene glycol and benzyl alcohol; glycerol formal, glycofural, polysorbate 80, decanol, 2-ethyl hexanol, ethyl acetate, butyl acetate, ethyl hexanoic acid, lactic acid, caproic acid, peppermint oil and dimethyl sulphoxide.

18. The pharmaceutical composition according to claim 17, wherein the solubilizer is benzyl alcohol or ethanol.

19. A method of preparing a water-free pharmaceutical composition, comprising
(i) providing a lipid vehicle comprising long-chain triglycerides (LCT) and at least 10% by weight of medium-chain monoglycerides (MCM), wherein the composition has a solid fat content (SFC) that is 40 to 60% at room temperature, 10 to 40% at body temperature, and essentially 0%;
(ii) heating the lipid vehicle provided in step (i) to a temperature where it has essentially 0% SFC and a liquid appearance; and
(iii) admixing the so heated lipid vehicle with a preparation, free from any solubilizer, of one or local anaesthetics in an anaesthetically effective amount.

20. A method according to claim 19, comprising the step of subjecting the mixture resulting from step (iii), under conditions where it has essentially 0% SFC, to pass through a filter having a pore size sufficient to sterilize the mixture.

21. A method of sterilizing a composition according to claim 1, comprising:
(i) heating the composition the composition to a temperature where it has essentially 0% SFC; and
(ii) subjecting so heated composition to pass through a filter having a pore size sufficient to sterilize the mixture.

22. A method of sterilizing a water free pharmaceutical composition comprising an at least partially lipid soluble pharmaceutical agent, comprising the steps of:
(i) providing a composition comprising the pharmaceutical agent; and a
a lipid vehicle comprising long-chain triglycerides (LCT) and at least 10% by weight of medium-chain monoglycerides (MCM), wherein the composition has a solid fat content (SFC) that is 40 to 60% at room temperature, 10 to 40% at body temperature, and essentially 0% at a temperature of below 60° C., preferably at a temperature of about 50 to 55° C.;
(ii) heating the composition to a temperature where it has essentially 0% SFC; and
(iii) subjecting so heated composition to pass through a filter having a pore size sufficient to sterilize the mixture.
